(19) United States
(12) Patent Application Publication
(10) Pub. No.: US 2022/0193387 A1
(43) Pub. Date: Jun. 23, 2022

(54) Ergonomic Tattoo Cartridge

(71) Applicant: Joseph B. Clark, Nine Miles Falls, WA (US)

(72) Inventor: Joseph B. Clark, Nine Miles Falls, WA (US)

(21) Appl. No.: 17/127,997

(22) Filed: Dec. 18, 2020

(51) Int. Cl. A61M 37/00

(52) U.S. Cl. CPC A61M 37/0084

(57) ABSTRACT

A tattoo cartridge that includes opposing radial cutouts to allow a user's thumb and forefinger to rest securely in a non-slip manner on both sides of the cartridge. Additionally, the side walls of the cartridge include raised diamond shaped ridges for further grip control. Indices are molded into the side wall of the cartridge to easily identify the cartridge.

### CROSS REFERENCE TO RELATED APPLICATIONS

Not Applicable

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of tattooing equipment and more specifically to an ergonomic tattoo cartridge.

Various embodiments of tattooing cartridges that are available and in use do not provide a user with a comfortable and slip resistant grip.

### BRIEF SUMMARY OF THE INSTANT INVENTION

The primary object of the invention is to provide a tattoo cartridge that is comfortable for the user to hold and ensures grip by providing a grip surface that prevents unintended user hand slippage during use.

Yet another object of the invention is to provide a tattoo cartridge that has a molded in labeling system that tells the user what type of cartridge is being used.

Other objects and advantages of the present invention will become apparent from the following descriptions, taken in connection with the accompanying drawings, wherein, by way of illustration and example, an embodiment of the present invention is disclosed.

The present invention relates to tattoo cartridges and more specifically to an ergonomic grip portion for a tattoo cartridge.

Tattoo cartridges are used in the tattooing process, which is a process of depositing colored ink under the top layer of skin of a person forming a decorative design or written statement. Tattoo cartridges are attached to a tattoo machine via a bayonet, or quarter turn connection. Each cartridge contains a needle. Some needles are used for fine line work. Other needles are used for shading. Before the advent of cartridges, the user would need to have a separate tattoo machine for each needle. With the cartridge system, the tattoo artist needs only one machine and can switch out needle cartridges as needed.

A disadvantage to current tattoo cartridges is that they can slip during use, especially when spending long periods of time doing intricate and/or large tattoos. Therefore, there is a need for an ergonomically comfortable grip portion for a tattoo cartridge that allows the user to hold onto the cartridge for prolonged periods of time without his or her hand slipping or experiencing muscle fatigue.

In accordance with a preferred embodiment of the invention, there is disclosed a novel tattoo cartridge, said cartridge being roughly cylindrical in shape and having right side and left side radial cutouts, said cartridge terminating at one end in a standard bayonet type connection means and at the other end in an orifice that allows a tattoo needle or needles to protrude through, said tattoo cartridge also having molded in indices that alert the user as to the type of cartridge being used, said tattoo cartridge approximately cylindrical shape with said side radial cutouts and also including diamond shaped raised portions on the remaining portions of said cylinder to further improve the user's grip of said tattoo cartridge.

### BRIEF DESCRIPTION OF THE DRAWINGS

The drawings constitute a part of this specification and include exemplary embodiments to the instant invention, which may be embodied in various forms. It is to be understood that in some instances various aspects of the instant embodiments may be shown exaggerated or enlarged to facilitate an understanding of the instant embodiment.

FIG. 1 is a perspective view of the invention.

FIG. 2 is a front view of the invention.

FIG. 3 is a side view of the invention.

FIG. 4 is a perspective view of the invention in use.

FIG. 5 is a perspective view of a second embodiment of the invention.

FIG. 6 is an exploded view of the second embodiment of the invention.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

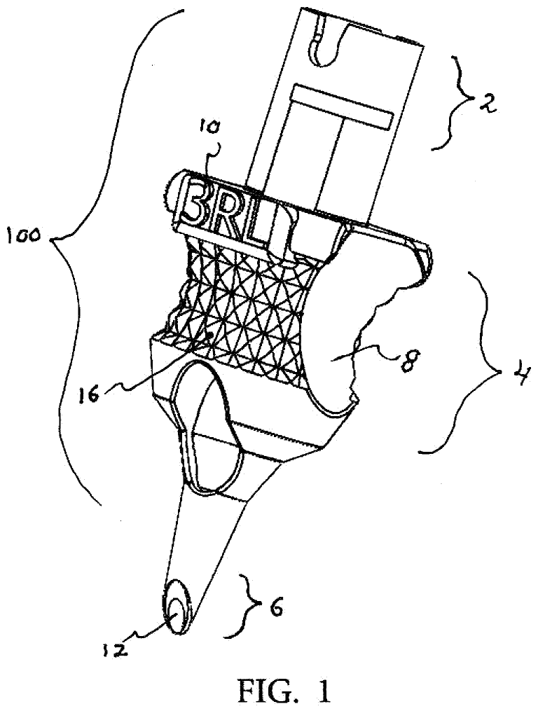

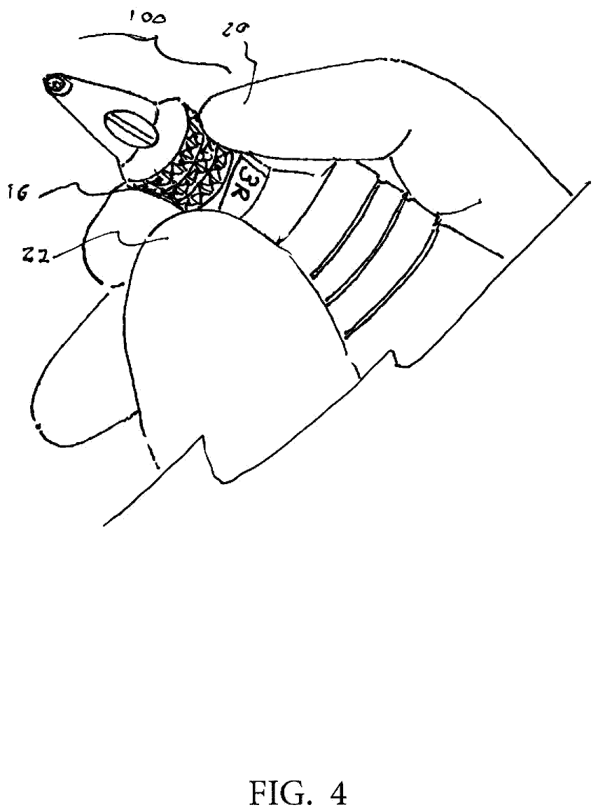

A perspective view of the tattoo cartridge of the present invention 100 is depicted in FIG. 1. The cartridge 100 is essentially cylindrical in shape in its central area 4 and has opposing radial cutout areas 8, 14 that are ergonomically designed to increase the user's comfort and control as he or she grasps the cartridge 100 during use as shown in FIG. 4. The additional raised diamond shaped surfaces 16 further aid the user in gripping the cartridge without slipping, especially when wearing protective gloves, as is generally the case. Molded in indices 10 alert the user as to the type of cartridge that is being used or will be used. The fact that the indices are molded into the structure of the cartridge means that they cannot be rubbed off or accidentally removed. The upper portion 2 of the cartridge 100 attaches to a standard tattoo tool via a standard bayonet or quarter turn fasting method. The conical needle extension bottom portion 6 of the cartridge 100 includes an aperture 12 to allow tattoo needle to extend beyond the cartridge 100 and deliver ink to a client's skin. In the preferred embodiment, the cartridge is manufactured using biodegradable material such as amorphous plastic which can more quickly degrade over time in comparison to other types of thermoformed plastics.

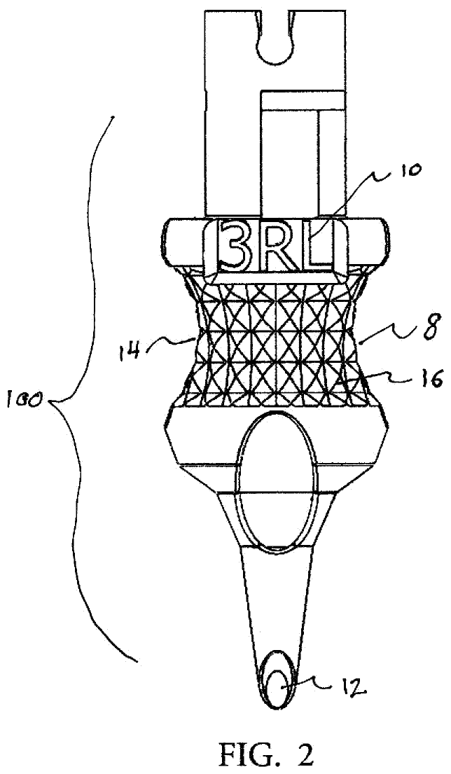

The front view of the cartridge 100 depicted in FIG. 2 is clearly showing the opposing radial cutout areas 14, 8 that are specifically designed to retain the user's thumb and forefinger.

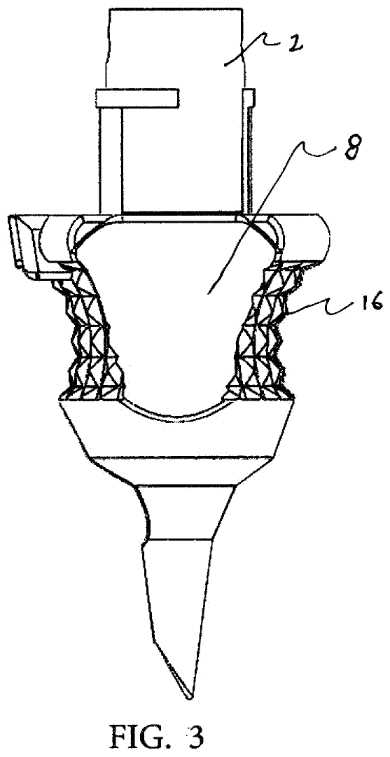

FIG. 3 is a side view of the cartridge 100 is clearly showing the shape of the radial cutout 8 and the profile of the raised, diamond shaped ridges 16.

Shown in FIG. 4 is a perspective view of the cartridge 100 being held by the user's thumb 22 and forefinger 20 while wearing a protective glove. The combination of the radial cutouts 8, 14 and diamond shaped raised surface 16 allow the user to have greater control of the cartridge 100 than other cartridges currently in use.

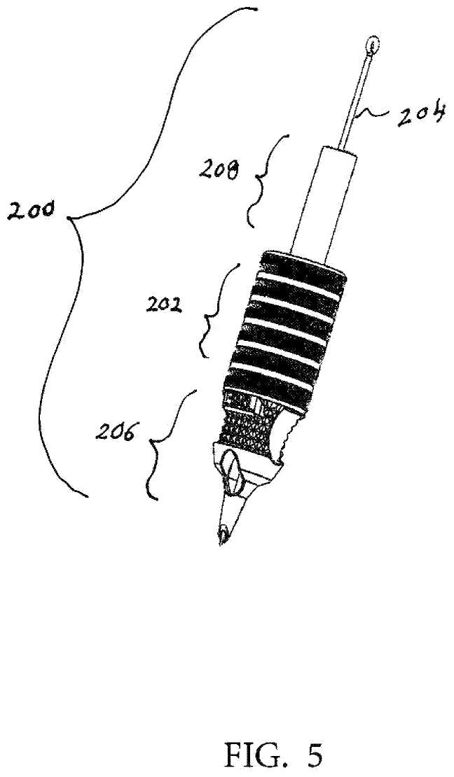

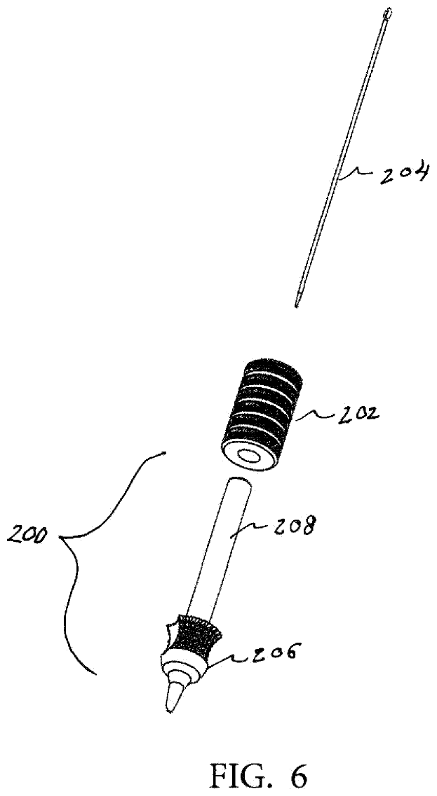

FIG. 5 is a perspective view of a second embodiment of the invention 200. The cartridge 200 as shown in the exploded view in FIG. 6 includes an additional cylindrical extension that centrally rises from the top of the lower section 206. Section 206 includes the identical ergonomic features as found in the first embodiment 100. The second embodiment 200 can be used with a rotary style, or coil grip tool. The outer cylindrical portion 202 has a centrally located aperture that allows the cylindrical extension 208 to slide into the cylindrical portion 202. A needle 204 extends through an aperture in cylindrical extension 208 and terminates at the bottom of lower section 206.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. A tattoo cartridge comprising:
a top portion, wherein the top portion further comprises a bayonet connection construction to attach the tattoo cartridge to a standard tattoo machine;
an essentially cylindrical central portion;
an essentially conical needle extension bottom portion, wherein the conical needle extension bottom portion comprises a needle protrusion aperture; and
the cylindrical central portion comprising a surface, wherein:
the cylindrical central portion surface comprises opposing radial cutouts for secure holding by a user's thumb and forefinger; and
the cylindrical central portion surface further comprises diamond shaped ridges, wherein the diamond shaped ridges increase the user's grip on the tattoo cartridge by making the cylindrical central portion surface less slippery.

2. A tattoo cartridge as claimed in claim 1 wherein the cylindrical central portion surface further comprises molded in tattoo cartridge identifying indices.

3. A tattoo cartridge as claimed in claim 1 wherein the cartridge is thermoformed using biodegradable amorphous plastic.

4. A tattoo cartridge as claimed in claim 1 comprising:
a needle, the needle comprising a cartridge end and a coil grip tool end, wherein the cartridge end is terminated at the conical bottom portion and the coil grip tool end terminates in a coil grip tool;
a tubular extension attached to the bottom portion of the tattoo cartridge, wherein the tubular extension comprises an axial tubular hollow, wherein the needle is located in said axial tubular hollow; and
a cylindrical central portion tubular extension, wherein the tubular extension with the needle is located in said cylindrical central portion tubular extension.
